(19) United States
(12) Patent Application Publication
(10) Pub. No.: US 2015/0283369 A1
(43) Pub. Date: Oct. 8, 2015

(54) Micro needle for transporting fluid across or into a biological barrier and method for producing such a micro needle

(71) Applicant: AMBRO B.V., Den Ham (NL)

(72) Inventors: Bouke Jan Brouwers, Almelo (NL); Arnoldus Maria Brouwers, Den Ham (NL)

(73) Assignee: Ambro B.V., Den Ham (NL)

(21) Appl. No.: 14/439,451

(22) PCT Filed: Oct. 3, 2013
(86) PCT No.: PCT/NL2013/050703

§ 371 Date: Apr. 29, 2015

(30) Foreign Application Priority Data
Nov. 13, 2012 (NL) 2009799

(51) Int. Cl. A61M 37/00

(52) U.S. Cl. CPC A61M 37/0015; A61M 2037/003; A61M 2037/0053

(57) ABSTRACT

The invention relates to a micro needle (1) for transporting fluid across or into a biological barrier. The micro needle comprises a shaft (2) having a hollow channel (3) ending in a tip (4) having a bevel at a predetermined angle. According to the invention the shaft is provided with an indentation (5) starting at the tip and running in longitudinal direction over a part of the outer surface having the shortest shaft length such that the channel has an unround cross section in the tip region. The invention further relates to a method for producing a micro needle according to the invention.

The present invention relates to a micro needle for transporting fluid across or into a biological barrier, wherein the micro needle comprises a shaft having a hollow channel ending in a tip having a bevel at a predetermined angle.

The micro needle approach shows clear advantages over competing methods of transferring fluids through skin or other biological barriers. In contrast to hypodermic needles, micro needles are relatively painless and can be self administered or administered by non-professionals. Furthermore, when using micro needles, only 10-20% of the drug or vaccine is needed compared to hypodermic needles. In addition, they overcome the molecular size limitations characteristic of conventional transdermal patches.

An array of micro needles according to the present invention is specifically suitable for use in a system described in the Dutch patent application NL 2007461 of the same applicant, which is not yet published. Said system comprises a displacement mechanism with ram and ensures penetrating the corneum stratum by hollow micro needles at a sufficient rate until a controlled depth is reached as well as raising the pressure in the capsule for pressing the fluid through the hollow micro needles.

In addition thereto said system provides a compact design with an elegant and simple operation. The system is patient friendly and allows for self-injection. It is thus suitable for daily use by any individual. It is also suitable for use in large groups, particularly in case of vaccination and even in case of urgent calamities, for instance due to an outbreak of a lethal virus, such as sars, ebola etcetera.

A micro needle according to the preamble is known in the art. US patent application 2008/0269666 describes a micro needle having a bevelled tip with a side-opening bore having an oval geometry. In the hollow embodiments the known micro needle is made of glass or polymer. The international application WO2010/051551 discloses a micro needle having a multi layered bevelled tip with a side-opening. Both known micro needles may in use break at the tip as a consequence of which small particles will remain in the biological barrier which may cause slow healing of the wound and may even lead to infections.

The invention has for its object to provide a micro needle according to the preamble that lifts this drawback.

According to the invention the micro needle is characterized in that the shaft is provided with an indentation starting at the tip and running in longitudinal direction over a part of the outer surface having the shortest shaft length such that the channel has an unround cross section in the tip region.

The unround cross section caused by the indentation leads to a robust tip that will not easily break. In a capsule or a patch multiple micro needles are present and with the micro needle according to the invention an even distribution of flow of the fluid into the biological barrier is guaranteed. This improved flow distribution over the micro needles can be explained by the restriction formed by the unround cross section. Furthermore the specific shape of the tip of the micro needle according to the invention ensures that in use layers of the biological barrier pierced by the micro needle tip remain attached and with human patients the microneedle will leave an incision in the skin rather than a cut-out. Hereby the occurrence of inflammation due to lose pieces of biological barrier remaining in the wound is effectively avoided.

It is noted that in FR2757405 or U.S. Pat. No. 2,560,162 injection needles are disclosed having an inward wall, respectively an inward depression, at the heel of the faceted channel outlet. The known needles are not suitable for use as micro needles for subcutaneous injection, more specifically intradermal injection in a biological barrier. Furthermore the dimensions of the inward wall or depression are in the order of the dimensions of the faceted edges present around the channel outlet and do not form any significant restriction to the flow.

In a first preferred embodiment the indentation at the tip has a depth larger than half of the outer diameter of the shaft at the end of the indentation, preferably between 65% and 95% of the outer diameter of the shaft at the end of the indentation. Preferably the indentation is at an indentation angle between 10 and 60 degrees, preferably 20-30 degrees, to the shaft. Preferably the indentation has a length greater than the channel opening, as seen in longitudinal direction of the shaft. Preferably the indentation has a length between 200% and 500% of the outer diameter of the shaft at the end of the indentation. Preferably the predetermined bevel angle lies between 20 and 80 degrees to the shaft, and preferably is 55-70 degrees. Due to these dimensions the insertion depth at which leakage is effectively prevented by the biological barrier closing off the opening of the tip lies between 100 and 300 micrometer. The micro needle according to this preferred embodiment is therefore ideal for subcutaneous injection, more specifically for intradermal injection.

In a preferred embodiment the width of the indentation increases in the direction of the tip. In a further preferred embodiment the shaft has a stepped shape in longitudinal direction. Both features attribute to decreasing dimensions of the channel diameter in the direction of the tip thus further improving flow characteristics.

Preferably at the tip the width of the indentation is such that the shaft is deformed over about half of its circumference, such that the non deformed part of the circumference forms the cutting edge of the needle. It is noted that in the injection needles known from FR2757405 or U.S. Pat. No. 2,560,162 the entire circumference cuts through the material.

The invention also relates to a method according to the preamble of claim 10, wherein the method comprises the following steps:

a) Deep-drawing a flat material into a micro needle comprising a shaft having a hollow channel ending in a tip;

b) Cutting the tip of the micro needle at a predetermined bevel angle to the shaft;

and is characterized by the step of:

c) Folding the shaft at the tip over part of the outer surface having the shortest shaft length inward over a distance of more than half of the outer diameter of the shaft, such that the channel has an unround cross section in the tip region. The method according to the invention directly results in a micro needle having a cutting edge formed by part of the circumference of the shaft at the tip. No additional polishing or sharpening is necessary contrary to FR2757405 or U.S. Pat. No. 2,560,162, wherein needles are faceted.

It is noted that in US 2008/0269666 hollow micro needles are made of glass using micropipette techniques or by casting polymer in a mold to make replicate arrays.

Preferably the folding is performed over a distance between 65% and 95% of the outer diameter of the shaft. More preferably the folding is preformed such that an indentation is made at an indentation angle between 10 and 60 degrees, preferably 20-30 degrees, to the shaft. Even more preferably the folding is performed such that an indentation is made having a length between substantially 100 and 1000 micrometer, preferably 400 -600 micrometer. Preferably at the tip the shaft is folded over about half of its circumference. The advantages of the preferred method steps have been discussed above.

The invention will now be elucidated in more detail herein below with reference to the drawings, in which:

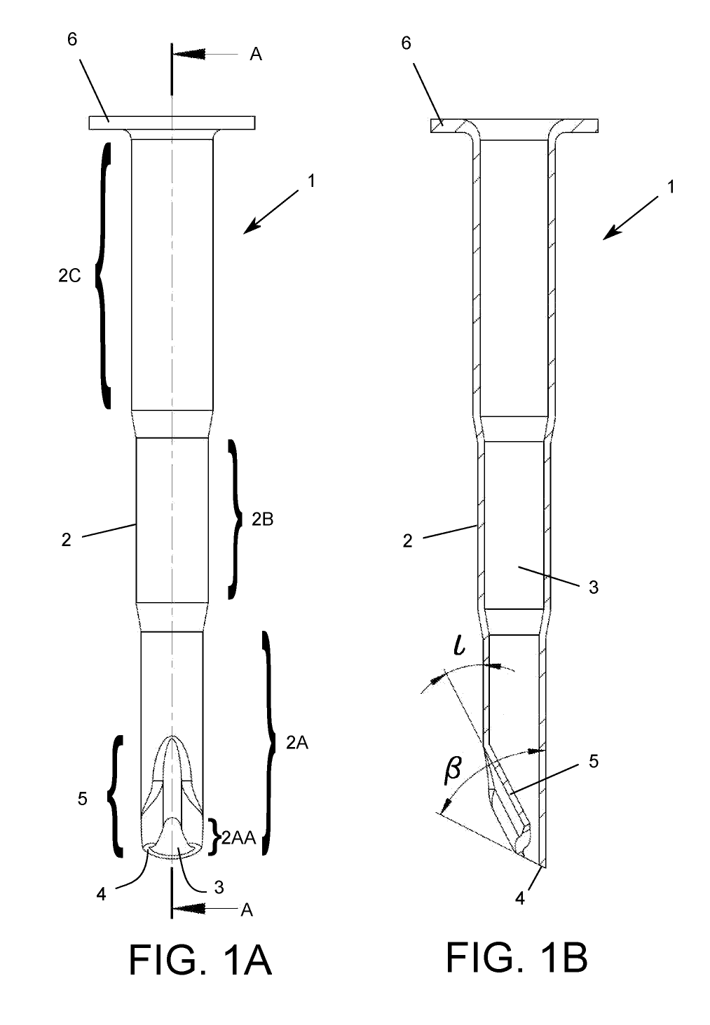

FIG. 1A shows a preferred embodiment of the micro needle according to the invention in a schematic view;

FIG. 1B shows the micro needle of FIG. 1A in longitudinal cross section;

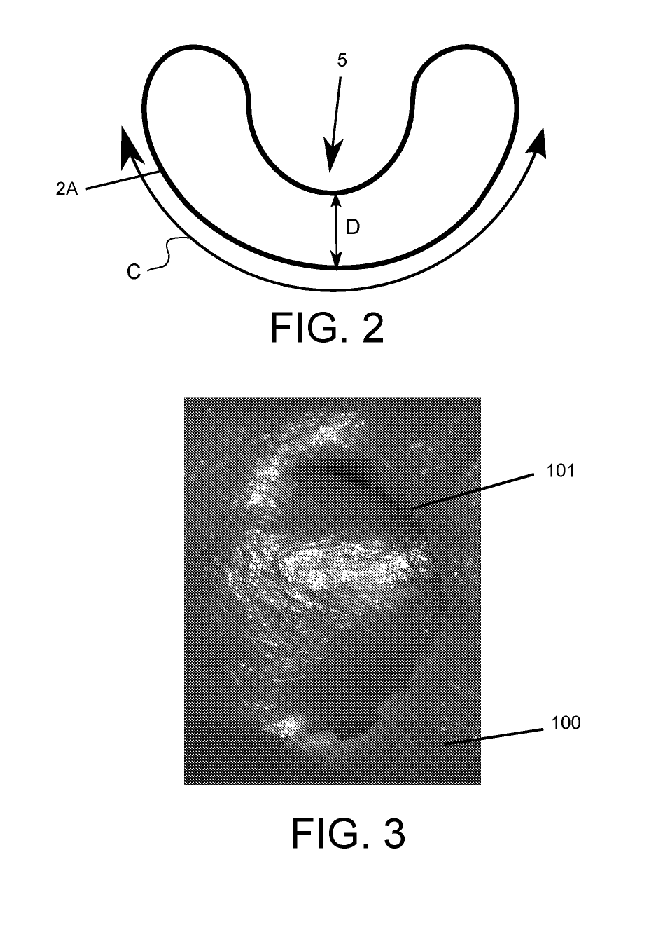

FIG. 2 shows a schematic view of a cross section of the tip of the micro needle of FIGS. 1A and 1B;

FIG. 3 shows a photograph of a test performed with the micro needle according to the invention;

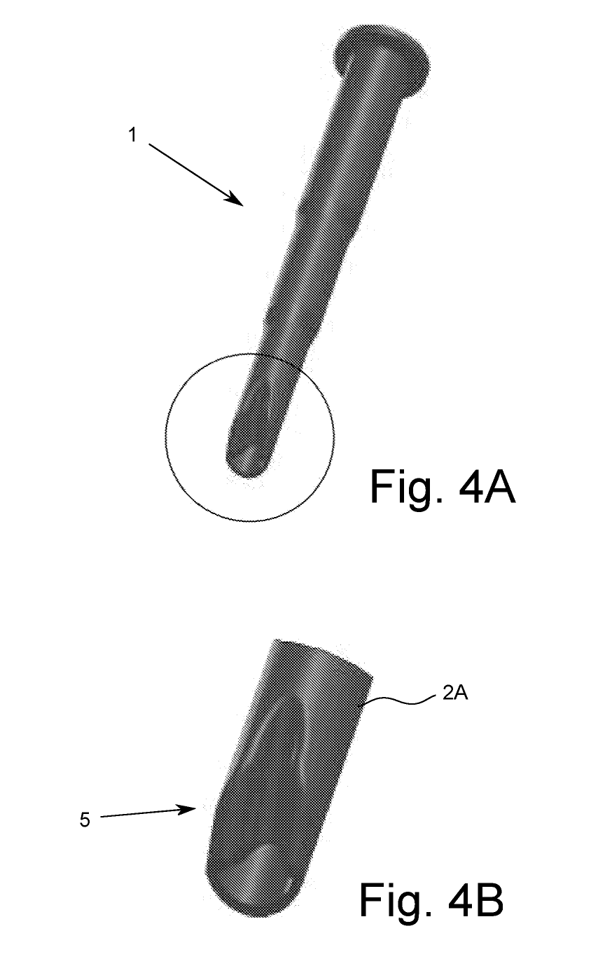

FIG. 4A shows a photographic image of the micro needle according to the invention; and

FIG. 4B shows the tip region of the microneedle in the photographic image of FIG. 4A in more detail.

FIG. 1A, FIG. 1B and FIG. 2 show respectively a schematic view and a longitudinal cross section of a micro needle 1 and a cross section of the tip of the micro needle 1 according to a preferred embodiment of the invention. Micro needle 1 comprises a longitudinal shaft 2 extending between a base 6 and a tip 4. The shaft 2 forms the body of the micro needle 1 and comprises a hollow channel 3 extending throughout the micro needle 1.

According to the invention the shaft 2 is provided with an indentation 5 near the tip 4. Indentation 5 starts at the tip 4 and runs in longitudinal direction over a part of the outer surface of the shaft, which part has the shortest shaft length. The indentation is such that the channel 3 has an unround cross section in the area of the indentation.

The indentation 5 preferably is at an indentation angle ι between 10 and 60 degrees, more preferably 20-30 degrees, with respect to the shaft 2.

Preferably the length of the indentation 5 is between substantially 100 and 1000 micrometer, more preferably 400-600 micrometer.

As can be seen in FIG. 2 in the region of the indentation the outer surface of the shaft is concave whereas the remainder of the outer surface of the shaft is convex. In other words the channel 3 has a generally kidney shaped cross section in the region of the indentation.

The distance D between the concave and convex surfaces is smaller than half of the outer diameter of the shaft before it was folded inward. The cutting edge C is smaller than half of the outer circumference of the shaft.

The tip 4 is a bevelled tip. The bevel is at a bevel angle β that preferably lies between 20 and 80 degrees with respect to the shaft 2. More preferably the bevel angle β is 55-70 degrees.

The length of the part 2AA defines the insertion depth necessary to seal off the channel 3 to prevent leakage. Part 2AA can also be referred to as the channel opening. In the preferred embodiment this part 2AA can be kept very small, in the order of substantially 200 micrometer, rendering the micro needle 1 specifically useful for subcutaneous injections and even intradermal injections.

Generally the diameter of the channel 3 decreases from the base 6 to the tip 4. Even at the tip this decreasing diameter is affected by the width of the indentation 5 that increases in the direction of the tip 4. The generally stepped shape in longitudinal direction of the shaft 2 also attributes to the decreasing diameter.

In the preferred embodiment shown the shaft 2 can be roughly divided into four parts. Part 2C is denoted as the base part. Part 2B is denoted as the middle part. Part 2A is denoted as the tip part. Part 2AA is denoted as the part minimally to be inserted in the biological barrier. Part 2C has the largest channel diameter. Part 2B has a smaller channel diameter than part 2C. Part 2A has a smaller channel diameter than part 2B.

FIG. 3 shows a photograph of a test performed with the micro needle according to the invention. The micro needle is inserted into an animal sample 100. It is clearly shown that the micro needle only leaves a small incision 101. The incision 101 has a curved shape corresponding to the convex outer surface of the shaft part 2AA. Clearly no part of the sample is cut out and thus no loose pieces are created.

According to the invention the following method for producing the micro needle 1 according to the invention can be used. In a first step a suitable flat material is converted into a micro needle comprising a shaft having a hollow channel ending in a tip by deep-drawing techniques. Suitable deep-drawing techniques are known in the art. Suitable materials are preferably metals, such as medical grade steel.

In a following step the tip 4 of the micro needle 1 is cut at a predetermined bevel angle β to the shaft 2. Suitable cutting techniques in combination with the known deep-drawing techniques are available in the relevant art.

The inventive step of the production method relates to denting the shaft 2 at the tip 4 over part of the outer surface having the shortest shaft length, such that the channel 3 has an unround cross section in the tip region 2A.

According to the invention the step of denting is performed such that the channel has a generally kidney shaped cross section in the tip region 2A. The denting is preferably performed such that an indentation 5 is made at an indentation angle ι between 10 and 60 degrees, preferably 20-30 degrees, to the shaft 2. Preferably the indentation 5 has a length between substantially 100 and 1000 micrometer, more preferably 400-600 micrometer.

The denting step can be performed by pushing a suitable tool against the tip region 2A. A pivoting movement of the tool may also be used. Another way of describing the step of denting may be folding the material of the shaft over a certain length. At the tip the shaft is folded inward over a distance of more than half of the outer diameter of the shaft. At the tip the folded part extends over about half of the outer circumference. The other half of the outer circumference forms the cutting edge of the micro needle. The indentation or inward curve squeezes the resulting channel outlet. In the preferred embodiment shown in FIG. 4 the outflow surface is reduced by more than 60%.

The micro needles can be constructed from a variety of materials. Preferred materials of construction include pharmaceutical grade stainless steel, gold, titanium, nickel, iron, tin, chromium, copper, palladium, platinum, alloys of these or other metals.

The length of the micro needles is selected for the particular application, accounting for both an inserted and uninserted portion. In transdermal applications, the “insertion depth” of the micro needles is preferably such that insertion of the micro needles into the skin does not penetrate into the dermis, thereby avoiding contacting nerves which may cause pain. In such applications, the actual length of the micro needles typically is longer, since the portion of the micro needles distal the tip lies in the substrate and cannot be inserted into the skin; the uninserted length depends on the particular device design and configuration. The actual (overall) height or length of micro needles should be equal to the insertion depth plus the uninserted length and may be about three to four millimetres.

The invention is of course not limited to the described and shown preferred embodiment. The invention relates generally to any embodiment falling within the scope of protection as defined in the claims and as seen in the light of the foregoing description and accompanying drawings.

1-16. (canceled)

17. A micro needle for transporting fluid across or into a biological barrier, wherein the microneedle comprises a shaft having a hollow channel ending in a tip having a bevel at a predetermined angle, wherein the shaft is provided with an inwardly folded part starting at the tip and running in longitudinal direction over a part of the outer surface having the shortest shaft length such that the channel has an unround cross section in the tip region.

18. The micro needle according to claim 17, wherein the inwardly folded part at the tip has a depth larger than half of the outer diameter of the shaft at the end of the inwardly folded part.

19. The micro needle according to claim 18, wherein the inwardly folded part at the tip has a depth between 65% and 95% of the outer diameter of the shaft at the end of the inwardly folded part.

20. The micro needle according to claim 17, wherein the inwardly folded part is at an indentation angle between 10 and 60 degrees to the shaft.

21. The micro needle according to claim 20, wherein the inwardly folded part is at an indentation angle between 20 and 30 degrees to the shaft.

22. The micro needle according to claim 17, wherein the predetermined bevel angle lies between 20 and 80 degrees to the shaft.

23. The micro needle according to claim 22, wherein the predetermined bevel angle lies between 55 and 70 degrees to the shaft.

24. The micro needle according to claim 17, wherein the inwardly folded part has a length greater than the channel opening.

25. The micro needle according to claim 17, wherein the inwardly folded part has a length between 200% and 500% of the outer diameter of the shaft at the end of the inwardly folded part.

26. The micro needle according to claim 17, wherein the inwardly folded part has a length between 100 and 1000 micrometers.

27. The micro needle according to claim 27, wherein the inwardly folded part has a length between 400 and 600 micrometers.

28. The micro needle according to claim 17, wherein the width of the inwardly folded part increases in the direction of the tip.

29. The micro needle according to claim 17, wherein at the tip the width of the inwardly folded part is such that the shaft is deformed over about half of its circumference, wherein the non-deformed part of the circumference forms the cutting edge of the needle.

30. The micro needle according to claim 17, wherein the shaft has a stepped shape in longitudinal direction.

31. A method for producing a micro needle for transporting fluid across or into a biological barrier wherein the method comprises the following steps:
a) deep-drawing a flat material into a microneedle comprising a shaft having a hollow channel ending in a tip;
b) cutting the tip of the microneedle at a predetermined bevel angle to the shaft; and
c) folding the shaft inward at the tip over part of the outer surface having the shortest shaft length over a radial distance of more than half of the outer diameter of the shaft, such that the channel has a nonround cross section in the tip region.

32. The method according to claim 31, whereby the folding is performed over a radial distance between 65% and 95% of the outer diameter of the shaft.

33. The method according to claim 31, whereby the folding is performed such that an inwardly folded part is made at an indentation angle between 10 and 60 degrees to the shaft.

34. The method according to claim 33, whereby the folding is performed such that the inwardly folded part is made at an indentation angle between 20 and 30 degrees to the shaft.

35. The method according to claim 31, whereby the folding is performed such that an inwardly folded part is made having a length between 100 and 1000 micrometers.

36. The method according to claim 35, whereby the folding is performed such that the inwardly folded part is made having a length between 400 and 600 micrometers.

37. The method according to claim 31, whereby at the tip the shaft is folded over about half of its circumference.
